(19) United States
(12) Patent Application Publication
(10) Pub. No.: US 2014/0171799 A1
(43) Pub. Date: Jun. 19, 2014

(54) SYSTEMS AND METHODS FOR PROVIDING ULTRASOUND PROBE LOCATION AND IMAGE INFORMATION

(71) Applicant: GENERAL ELECTRIC COMPANY, SCHENECTADY, NY (US)

(72) Inventors: JOHN ERIK HERSHEY, BALLSTON LAKE, NY (US); MICHAEL JAMES HARTMAN, CLIFTON PARK, NY (US); PIERINO GIANNI BONANNI, LOUDONVILLE, NY (US); STEPHEN FRANCIS BUSH, LATHAM, NY (US); MICHAEL JOSEPH DELL'ANNO, CLIFTON PARK, NY (US); STANISLAVA SORO, NISKAYUNA, NY (US)

(73) Assignee: GENERAL ELECTRIC COMPANY, SCHENECTADY, NY (US)

(21) Appl. No.: 13/718,762

(22) Filed: Dec. 18, 2012

Related U.S. Application Data
(60) Provisional application No. 61/736,973, filed on Dec. 13, 2012.

(51) Int. Cl. A61B 8/00; A61B 8/13; A61B 5/00; A61B 8/08

(52) U.S. Cl. CPC A61B 8/4254; A61B 8/463; A61B 8/5261; A61B 8/5253; A61B 8/13; A61B 5/0077; A61B 5/0064; A61B 5/0035; A61B 8/4444; A61B 8/54; A61B 8/565; A61B 8/467; A61B 8/5207
USPC 600/440

(57) ABSTRACT

Systems and methods for providing ultrasound probe location and image information are provided. One system includes an ultrasound device coupled with an ultrasound probe and configured to acquire ultrasound images of a subject. The system further includes at least one of a plurality of digital cameras or a plurality of digital scanners configured to acquire scene information including images of the ultrasound probe with the subject during an image scan. The system also includes a processor having an ultrasound registration unit (URU) with the URU configured to identify and reference a probe location of the ultrasound probe to a surface of the object from the scene information and correlate the probe location to one or more of the acquired ultrasound images. The URU is additionally configured to generate a representation of the surface showing the identified and referenced probe location corresponding to the correlated ultrasound images.

### CROSS REFERENCE TO RELATED APPLICATIONS

This application claims priority to and the benefit of the filing date of U.S. Provisional Application No. 61/736,973 filed Dec. 13, 2012, the subject matter of which is herein incorporated by reference in its entirety.

### BACKGROUND

Remote health care services, such as performing diagnostic imaging in remote locations that otherwise may not have adequate health care facilities, are increasing. This increase is due in part because in a typical centralized medical care system arrangement, the transportation of patients to a centralized facility takes time, which can result in treating patients later in a disease pathology and can add cost.

For example, one arrangement for the healthcare practice is to perform healthcare services only in large centralized institutions such as major hospitals. Another arrangement is to provide healthcare services to the patient at the patient's location such as the patient's home or, ultimately, with the patient while the patient is “on the go.” The centralized approach is expensive and not always efficacious with respect to necessary patient care. The patient location approach also can be very expensive and similarly non-efficacious as modern medical testing often includes the use of technological implements such as imaging modalities, for example, ultrasound and x-ray devices, that require units too expensive to be deployed on a one-for-one patient basis. There is also the problem of conducting a proper exam as generally the patient will not have the skill or ability to perform a proper self-examination.

Accordingly, there is an increased development of systems to provide more effective healthcare services in a decentralized environment such as by many small and dispersed medical centers that are generally nearer the majority of remote patients than large medical centers. Additionally, these smaller centers may handle many patients instead of just one or a few.

In this decentralized remote health care area, a patient may be examined by a remote health care practitioner (RHCP) in a medical dispensary remote from a major medical center such as a hospital. The RHCP may perform a protocol for a diagnostic test and possibly some treatment under the guidance and supervision of a specialist located at the major medical center. Thus, a RHCP may conduct medical tests at a location remote from a large centralized medical facility such as a major hospital. The RHCP may be under the direction of a specialist, such as a doctor, located in a large centralized medical facility.

However, there are problems with this decentralized healthcare model. One shortcoming relates to modalities involving examination procedures wherein the details are difficult to accurately describe to the remote specialist. For example, an electrocardiogram is relatively straightforward to describe. In particular, the leads are positioned per instruction and the one-dimensional ECG data itself is straightforwardly communicated to the remotely located specialist. Some imagery data, however, such as is generated during an ultrasound examination, requires the RHCP to slide, rotate, tilt, compress, and/or rock the ultrasound probe transducer. Some of these movements may be satisfactorily communicated by orientation sensors located on the probe or by descriptive text, voice, or other metadata. The location of the probe on the patient's body surface is, however, both important and difficult to describe.

With conventional methods, such description of the probe location for an examination may not be accurately provided or not provided in a timely manner. Moreover, when the probe moves, for example, in elevation or rotates, there is almost no alignment and the alignment of the images becomes even more difficult. The lack of probe location information may lead to improper diagnosis or blurred and/or jagged images in the reconstruction process.

In one embodiment, an ultrasound imaging system is provided that includes an ultrasound device coupled with an ultrasound probe and configured to acquire ultrasound images of a subject. The ultrasound imaging system further includes at least one of a plurality of digital cameras or a plurality of digital scanners configured to acquire scene information including images of the ultrasound probe with the subject during an image scan. The ultrasound imaging system also includes a processor having an ultrasound registration unit (URU) with the URU configured to identify and reference a probe location of the ultrasound probe to a surface of the object from the scene information and correlate the probe location to one or more of the acquired ultrasound images. The URU is additionally configured to generate a representation of the surface showing the identified and referenced probe location corresponding to the correlated ultrasound images.

In another embodiment, a method for communicating probe location information synchronized with ultrasound image data is provided. The method includes obtaining ultrasound image data for a subject acquired by an ultrasound probe and obtaining scene information acquired by at least one of a plurality of digital cameras or a plurality of digital scanners, wherein the scene information includes images of the ultrasound probe with the subject during an image scan. The method further includes identifying and referencing a probe location of the ultrasound probe to a surface of the object from the scene information and synchronizing in time the probe location to one or more of the acquired ultrasound images. The method also includes generating a representation of the surface showing the identified and referenced probe location corresponding to the synchronized ultrasound images. The method additionally includes communicating the representation with the synchronized ultrasound images to a location remote from an ultrasound system controlling the ultrasound probe.

### BRIEF DESCRIPTION OF THE DRAWINGS

FIG. 1 is a schematic block diagram of an image communication system formed in accordance with an embodiment.

FIG. 2 is a diagram illustrating a camera within the image communication system of FIG. 1.

FIG. 3 is a diagram illustrating a mode of operation for an ultrasound examination in accordance with an embodiment.

FIG. 4 illustrates a patient with retro-reflective patches in accordance with one embodiment.

FIG. 5 is a diagram illustrating another mode of operation for an ultrasound examination in accordance with an embodiment.

FIG. 6 is a flowchart of a method for communicating probe location information synchronized with ultrasound image data in accordance with various embodiments.

FIG. 7 is a diagram illustrating a user interface in accordance with various embodiments.

FIG. 8 illustrates a hand carried or pocket-sized ultrasound imaging system formed in accordance with an embodiment.

FIG. 9 illustrates an ultrasound imaging system formed in accordance with an embodiment and provided on a moveable base.

FIG. 10 illustrates a 3D-capable miniaturized ultrasound system formed in accordance with an embodiment.

### DETAILED DESCRIPTION

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers, circuits or memories) may be implemented in a single piece of hardware or multiple pieces of hardware. It should be understood that the various embodiments are not limited to the arrangements and instrumentality shown in the drawings.

As used herein, an element or step recited in the singular and proceeded with the word “at” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments provide systems and methods for determining the position of an ultrasound probe on a body of a patient and synchronizing or correlating this information with acquired image data. By practicing various embodiments, a remotely located specialist can receive ultrasound probe location information synchronized with corresponding image data (e.g., image frames). At least one technical effect of various embodiments is improved information for images communicated from one location to a different second location.

Various embodiments provide an imaging system that communicates information, such as diagnostic images, from one location (e.g., a patient examination site) to another location (e.g., a hospital remote from the examination site) along with probe location information, which may be communicated over one or more communication channels. It should be noted that the images may be, for example, a streaming series or sequence of images over one or more communication channels. In one embodiment, for example, a remote health care practitioner (RHCP) may be guided by a specialist using the communicated information.

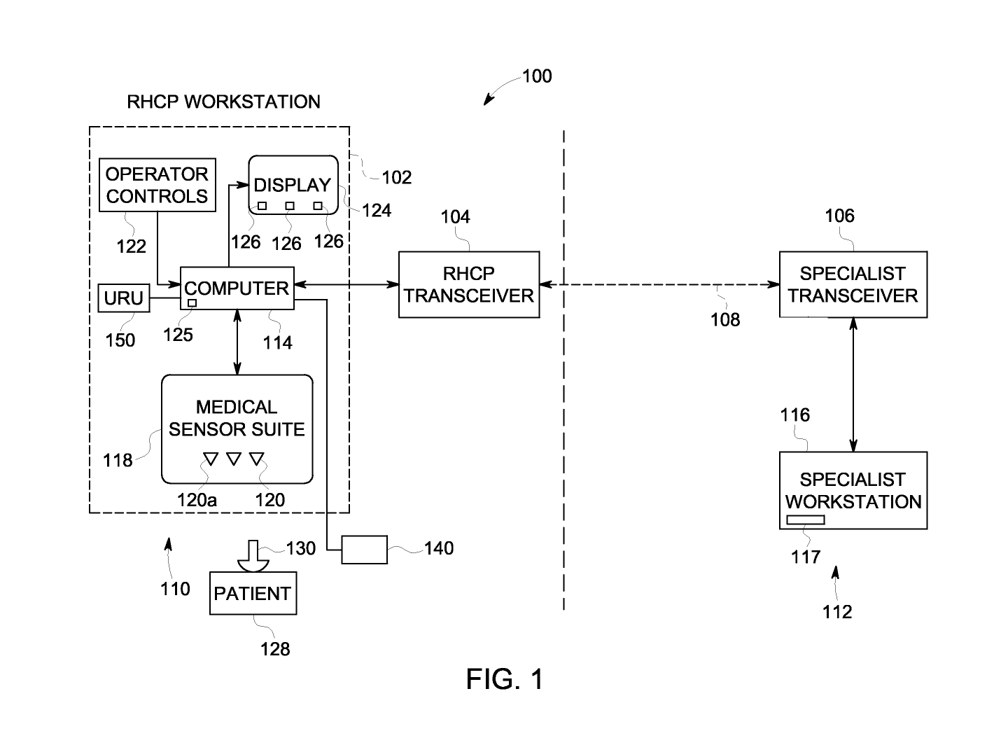

FIG. 1 is a schematic block diagram of an image communication system 100 for communicating image data in accordance with various embodiments. The image communication system 100 is generally configured to acquire medical images, such as ultrasound imagery (e.g., a plurality of ultrasound images over time) at the RHCP's location (as well as probe location information) and transmit that imagery and probe location information to, for example, a remotely located specialist for viewing, consultation and/or guidance, which may include providing feedback. The image communication system 100 includes an RHCP workstation 102 that allows acquisition of image data (and probe location information) and interface with a user or operator, such as the RHCP. It should be noted that although various embodiments are described in connection with communicating ultrasound data, the various embodiments may be used to communication other types of medical and non-medical image data, such as other types of medical images, diagnostic audio, electrocardiogram (ECG) and other physiological waveforms, which may be communicated in a streaming manner.

The system 100 includes an RHCP transceiver 104 that communicates with a remote transceiver, which in the illustrated embodiment is a specialist transceiver 106 (e.g., a transceiver located at a location of a specialist). The transceivers 104, 106 communicate over or form a communication link 108, which may include one or more communication channels (e.g., cellular network communication channels). Accordingly, the communication link 108 provides bi-directional or two-way communication between a first location 110 and a second location 112, which may be an examination location and a specialist location remote therefrom (e.g., miles away), respectively, in one embodiment.

With respect to the first location 110 where the image data is acquired and processed, the RHCP workstation 102 includes a processor, which is illustrated as a computer 114. The computer 114 is coupled to the RHCP transceiver 104 to allow communication between the computer 114 and another workstation at the second location 112, illustrated as a specialist workstation 116, via the specialist transceiver 106. It should be noted that the RHCP transceiver 104 and the specialist transceiver 106 may form part of or be separate from the RHCP workstation 102 and the specialist workstation 116, respectively. It also should be noted that the workstations 102 and 116 may be any types of workstations usable by different types of operators.

The computer 114 is also connected to one or more medical devices 120 illustrated as a medical sensor suite 119. The medical devices 120 may be removably and operatively coupled to an interface (now shown) of the RHCP workstation 102 to allow communication therebetween. The medical sensor suite 119 may include a plurality of different types or kinds of medical devices, such as plurality of different types of medical imaging probes that may be used for different imaging applications. In one embodiment, the medical device 120a is an ultrasound imaging apparatus that may be used to image a patient 128 or a portion of the patient 128.

The computer 114 is also coupled to a user input 122 that includes one or more user controls (e.g., keyboard, mouse and/or touchpad) for interfacing or interacting with the RHCP workstation 102. The computer 114 is also coupled to a display 124, which may be configured to display one or more ultrasound images 126, such as in a time sequence or loop of images, also known as a cine loop. In operation, a user is able to control the display of the images 126 on the display 124 using the user input 122, for example, controlling the particular display settings. The user input 122 may also allow a user to control the acquisition of the image data used to generate the images 126, such as the image acquisition settings or controls. In one embodiment, the user input 122 allows control of the ultrasound imaging apparatus 120a.

The ultrasound imaging apparatus is configured to acquire ultrasound image data that may be processed by the ultrasound imaging apparatus 120a or the RHCP workstation 102 to generate one or more images (e.g., 2D, 3D or 4D images) of a region of interest, for example an anatomy of interest, of a subject, such as the patient 128. The ultrasound imaging apparatus 120a or the RHCP workstation 102 generates one or more images by reconstructing imaging data acquired by the ultrasound imaging apparatus 120a. It should be noted that as used herein, imaging data and image data both generally refer to data that may be used to reconstruct an image.

In one embodiment, the imaging data is acquired with an imaging probe 130. The imaging probe 130 may be a hand-held ultrasound imaging probe. Alternatively, the imaging probe 130 may be an infrared-optical tomography probe. The imaging probe 130 may be any suitable probe for acquiring ultrasound images in another embodiment. The imaging probe 130 may be mechanically coupled to the ultrasound imaging apparatus 120a. Alternatively or optionally, the imaging probe 130 may be in wireless communication with the ultrasound imaging apparatus 120a. In still other embodiments, the imaging probe 130 is alternatively or optionally coupled to the RHCP workstation 102.

The computer 114 is further coupled to a camera 140, which in one embodiment, is a digital camera. For example, the camera 140 may communicate images with probe location information for synchronizing the location of the imaging probe 130 with one or more corresponding image frames acquired during an image scan by the ultrasound imaging apparatus 120a. For example, the camera 140 in various embodiments is configured to acquire “scene information”, which in various embodiments is a series of digital pictures of the examination scene, including the patient 128 and the probe 130 being used to acquire the ultrasound image data. The camera 140 may acquire digital pictures periodically (e.g., every 3, 5, 10 or 30 seconds) during the ultrasound scan. The camera 140 may be any suitable digital camera, for example, a camera having a defined minimum resolution level (e.g., 5 mega-pixels) and optionally optical or digital zoom capabilities. Is some embodiments, the camera 140 also allows for storage therein of the acquired scene images.

In operation, data acquired by the ultrasound imaging apparatus 120a and the camera 140 is accessible and may be communicated between the first location 110 and the second location 112 using the transceivers 104, 106. It should be noted that the transceivers 104, 106 may be configured to communicate using any suitable communication protocol, such as a suitable wireless communication protocol, for example cellular 3 G communication protocols. Using this arrangement, data from the computer 114 at the RHCP workstation 102 may be transmitted to a specialist at the specialist workstation 116 and data sent from the specialist may be received at the RHCP workstation 102.

Various embodiments provide for acquiring and communicating probe location information correlate or synchronized (e.g., synchronized in time) with the acquired image data. For example, is some embodiments, three different modes of operation may be provided. In particular, in a first mode (Mode 1), the output of a plurality of cameras 140 (e.g., digital cameras) is used to estimate the contour of the patient's body and the ultrasound probe's location on the patient's body. In a second mode (Mode 2), the output of a plurality of digital scanners 420 (shown in FIG. 5) is used to estimate the contour of the patient's body and the ultrasound probe's location on the patient's body. In a third mode (Mode 3), the output of a set of cameras 140 is processed by an ultrasound registration unit (URU) 150 with the output of a set of digital scanners 420 to estimate the contour of the patient's body and the ultrasound probe's location on the patient's body. It should be noted that the URU 150 may be coupled to or form part of the computer 114, such as a module. The URU 150 may be implemented in hardware, software, or a combination thereof.

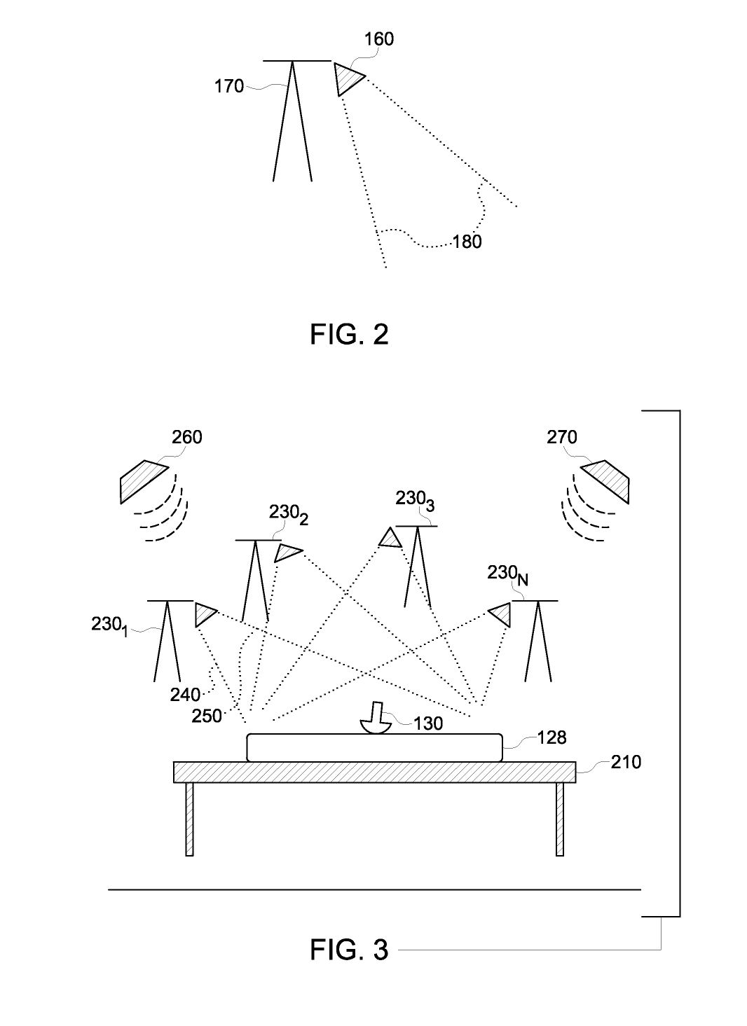

For an examination using Mode 1, digital cameras are set up in the medical area. For example, FIG. 2 illustrates a digital camera 160 (which may be embodied as the camera 140 shown in FIG. 1) on a support structure 170. The angular limit of the field of view of the camera is indicated by the dotted lines 180. The support structure 170 may be, for example, a camera stand or other suitable support.

The ultrasound examination facility used by the RHCP during a Mode 1 examination is illustrated in FIG. 3. As can be appreciated, this exam set up may be performed at a location remote from a specialist. The patient 128 lies on a support table 210. Illumination of the patient 220 may be provided by one or more light sources, illustrated as lamps 260 and 270. A set of N digital cameras 230₁, 230₂, 230₃, . . . , 230_{N} are positioned such that the fields of views of the digital cameras 230 overlap the patient 128, or regions of interest of the patient 128. The angular limits of the fields of view of the cameras 230 are indicated by the dotted lines from the cameras, shown as lines 240 and 250 for camera 230₁. The outputs of the N digital cameras (e.g., digital still images or digital movies) are communicated to the URU 150 (shown in FIG. 1), which may be communicated through a wired or wireless link.

Position information for the probe 130 (e.g., scene images showing the probe 130 in combination with or in contact with the patient 128) is also communicated to the URU 150 by one or more of the digital cameras 230 and the location of the probe is then referenced to the patient's body. As described in more detail herein, using the output images of the digital cameras 230 and the known location coordinates of the digital cameras 230 relative to the patient 128, a model of the body of the patient 128 may be generated and used to determine the location of the probe at the time image data was acquired.

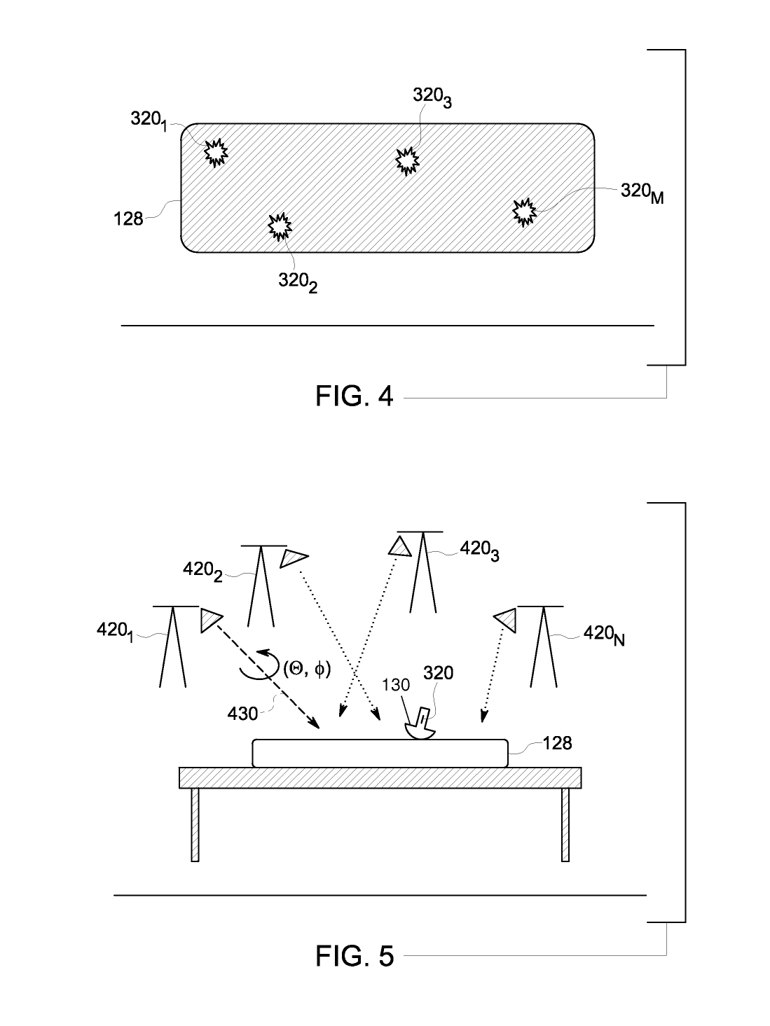

For an examination using Mode 2, the patient 128 is fitted with M retro-reflective patches 320₁, 320₂, 320₃, . . . , 320ₘ as illustrated in FIG. 4. For example, a plurality of retro-reflective patches 320 are coupled (e.g., taped) to the body of the patient 128 at determined locations, which may be evenly or unevenly distributed. The retro-reflective patches 320 may be any type of patches having reflective qualities when light is incident thereon. For example, the retro-reflective patches 320 may be formed from a reflective material that reflects light.

The ultrasound examination facility used by the RHCP during a Mode 2 examination is illustrated in FIG. 5. The patient 128 is fitted with the retro-reflective patches 320 as illustrated in FIG. 4. A set of N digital scanners 420₁, 420₂, 420₃, . . . , 420_{N} are positioned such that the fields of views of the digital scanners 420 overlap the patient 128 or a region of interest of the patient 128. The digital scanners 420 are operable to step a directed small spot size light field through a scan pattern characterized by a set of angles. For example, the digital scanner 420₁ is illustrated as emitting a small spot light field 430 at angles θ and φ. When a directed small spot size light field is aimed at one or more of the retro-reflective patches 320, a specular reflection takes place back along the direction of scan to the digital scanner 420 illuminating the retro-reflective patch 320 and the retro-reflection is detected by the digital scanner 420. The retro-reflection event is communicated to the URU 150 (shown in FIG. 1) along with the θ and φ at which the specular reflection was detected. It should be noted that the probe 130 also may be fitted with a retro-reflective patch 320 and the probe's position communicated to the URU 150 by one or more of the digital scanners 420. The location of the probe 130 is then referenced to the patient's body as described in more detail herein. It should be noted that the digital scanners 420 may be any device that projects light or light patterns, which may be along a defined scan path.

An examination under Mode 3 uses data from a set of digital cameras 230 fused with data from the digital scanners 420. Thus, this mode is a combination of Modes 1 and 2. It should be noted that the digital cameras 230 and/or digital scanners 420 in the various embodiments and modes may be supported and positioned in different locations, which may be movable depending on the support structure for the digital cameras 230 and/or digital scanners 420.

In operation, the URU 150 receives the outputs of the plurality of digital cameras 230 (Mode 1) and the location coordinates thereof, or the outputs of the plurality of digital scanners 420 (Mode 2) and location coordinates thereof, or the outputs from a set of digital cameras 230 and a set of digital scanners 420 (Mode 3) and the location coordinates thereof. The URU 150 uses this information to construct a model of the patient's body surface and prepare a representation of such surface. The ultrasound probe's location in reference to the patient's body (e.g., a scene image) is also reported to the URU 150 by one or more of the digital cameras 230 and scanners 420 and the probe's location is then referenced to the patient's body thereon to be sent to the remotely located specialist synchronized or correlated to and with the ultrasound imagery that was produced by the probe at that location. For example, the information from the digital cameras 230 and/or digital scanners 420 may be time stamped with the time stamp information then used to identify and correlate the image data acquired by the probe 130 to the corresponding location information, such that the information is synchronized in time.

More particularly, in Mode 1, the outputs of the digital cameras 230 are used to generate a best fit according to a specified norm. Specifically, a norm is a function that associates a strictly positive length with all non-zero vectors in a vector space. Examples of norms that may be used are the Euclidean norm, the Manhattan or Taxicab norm, or the general p-norm. This best fit can be done either by using a patient body surface model and fitting, according to the norm used, the parameters of the model to the scenes reported by the cameras (e.g., digital scene images), or the outputs of the cameras may be fused to yield an estimated body surface by minimizing the norm of the residuals in fitting the over-constrained problem that presents itself when N>3. Thus, in Mode 1 the probe location is recognized or determined only by the image or scene information (e.g., pictures of the patient 128 with the probe 130 during examination) acquired by the digital cameras 230 without the use of the retro-reflective patches 320. In this mode, the patient's body is localized using the images from the digital cameras 230 and the location of the probe 130 identified, such as using a shape matching algorithm to identify the patient 128 and/or the probe 130 in the scene pictures acquired by the digital cameras 230.

In Mode 2, the outputs of the digital scanners 420 are used to generate a best fit according to the specified norm. This best fit can be done by estimating the locations of the retro-reflective patches 320 and using these estimated locations as boundary conditions on a model of the patient's body surface and solving for the three-dimensional location of other points on the patient's body surface by interpolating between the estimated locations of the retro-reflective patches 230. As with Mode 1, the outputs of the scanners 420 may be fused to yield the estimated locations of the retro-reflective patches 230 by minimizing the norm of the residuals in fitting the over-constrained problem that presents itself when N>3.

In Mode 3 the camera information is combined with the scanner information. The camera information may be weighted differently from the scanner information and the computed norm uses the different weightings when minimizing residuals.

It should be noted that the location of the probe also may be supplemented using other devices. For example, probes with sensors that allow a determination of the magnetic orientation of the device may be used. As another example, accelerometers may be used in connection with the probe, for example, a three-axis accelerometer, a gyroscope, such as a three-axis gyroscope, or the like that determines the x, y, and z coordinates of the probe 130. As still another example, local location mechanisms or GPS (or the like) may be used. Thus, in some embodiments the probe 130 may include a sensor coupled therewith (e.g., a differential sensor). The sensor may be externally coupled to the probe 130 or may be formed integrally with and positioned in a housing of the probe 130 in other embodiments. The tracking device may receive and transmit signals indicative of a position thereof and is used to acquire supplemental positional data of the probe 130. For example, the sensor determines a position and an orientation of the probe 130. Other position sensing devices may be used, for example, optical, ultrasonic, or electro-magnetic position detection systems.

It should be noted that the locations for the digital cameras 230 in Mode 1 or the digital scanners 420 in Mode 2 or the digital cameras 230 and digital scanners 420 in Mode 3 is selected to reduce or minimize the likelihood that the RHCP blocks the view of one or more cameras 230 or scanners 420 during the ultrasound examination. Also, it should be noted that errors associated with the geometric dilution of precision (GDOP), a measure of the change of estimated target location with change in the measured data, is accounted for to have minimal effect on estimated target data. Accordingly, the locations of the digital cameras 230 with respect to the patient 128 in Mode 1 or the locations of the digital scanners 420 with respect to the patient 128 in Mode 2 or the locations of the digital cameras 230 and the digital scanners 420 in Mode 3 are selected according to at least two criteria in some embodiments. These criteria are that: (1) the probability that the RHCP will obscure more than one or two of the fields of view or fields of scan during the ultrasound examination is minimized, and (2) that each subset of the digital cameras 230 is placed with respect to the patient 128 so that the field of views of the digital cameras 230 with respect to the patient 128 essentially minimizes the GDOP. In the case of digital scanners 420, the placement of the scanners 420 is such that each subset of the scanners 420 is placed so that the GDOP of the scanners 420 with respect to the retro-reflective patches 320 on the patient is essentially minimized.

Variations and modifications are contemplated. For example, a mode of operation may be provided in which the model of the patient's body uses quantization that depends upon the magnitude of the norm of the residuals. In particular, the smaller the magnitude of the norm, the finer the quantization, and the larger the magnitude, the coarser the quantization.

In situations where the expert needs to effectively guide the RHCP in applying the ultrasound probe 130, the communication channel between the RHCP and expert may have significant latency that can affect attempts by the expert to verbally guide the location and application of the remote ultrasound unit during an exam, which is a time-delay control problem. By practicing various embodiments, the RHCP does not have to move the probe extremely slowly, waiting for verbal feedback before each motion.

In some embodiments, video from the exam may be buffered at the expert's location with ultrasound frame registration information. Ultrasound frame registration may be implemented via acoustically unique markers that are positioned at fixed locations around the patient 128, attached to the body, embedded on the table surface, or within a wearable patient accessory, so that the markers are easily identifiable within the ultrasound's field of view. The RHCP can perform an initial exam at normal speed while the ultrasound data is buffered at the expert side. The expert can review the registered frames looking for features of interest as well as the acoustic markers. The expert can guide the RHCP by reviewing the buffer and providing a buffered frame number (registered to a position), offset and orientation to a new location.

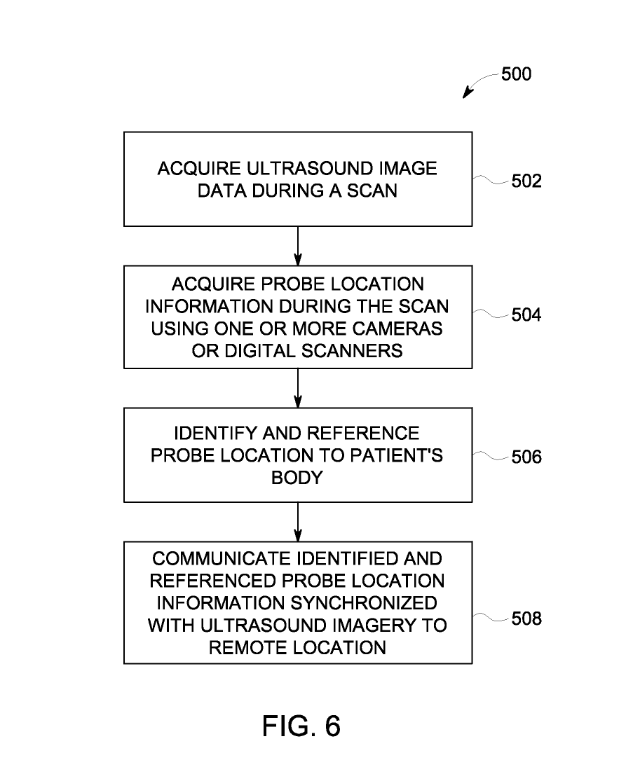

A flowchart of a method 500 in accordance with various embodiments for communicating probe location information synchronized with ultrasound image data is shown in FIG. 6. The method 500 allows for a determination of the location of the probe relative to the patient's body to be communicated to a remote location with the image data such that the probe location information corresponding to frames of ultrasound data are correlated or synchronized.

The method 500 includes acquiring at 502 ultrasound image data during a scan, for example, an ultrasound examination of a patient. The ultrasound image data may include acquiring ultrasound images using a determined scan protocol. During the scan, the operator may move (e.g., rotate or translate) the probe to acquire different views or image frames of a region of interest.

The method 500 also includes acquiring probe location information during the scan at 504. In various embodiments, the probe location information is acquired using a plurality of digital cameras or digital scanners (in combination with retro-reflective patches on the patient and optionally the probe). For example, during the scan, time stamped images of the patient and probe are acquired and stored. The time stamping of these digital scene images allows for correlation to the ultrasound image data acquired at 502.

Using the probe location information, the probe location during the scan is identified and referenced to the patient's body at 506. For example, using digital image information, which may be image scenes (e.g., images of the probe on the patient's body) and/or retro-reflective scanning, the body contour of the patient may be defined and fit to the probe location as described in more detail herein. For example, an over-constrained problem may be solved to determine the location of the probe along the contour of the patient corresponding to an acquired image frame.

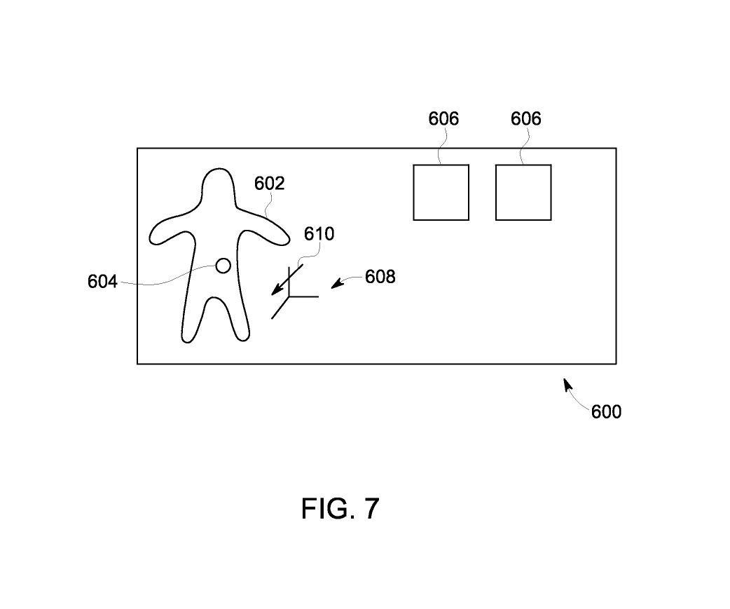

The identified and referenced probe location information correlated or synchronized with the ultrasound imagery is communicated to a remote location at 508. For example, a representation of the patient's body surface may be generated and displayed with a graphical indicator of the location of the probe along the surface of the body based on the fitting. For example, as shown in FIG. 7, a user interface 600 may be provided that is displayed at the remote location (e.g., on a specialist's workstation display). The user interface may include a two-dimensional representation 602 of the patient, such as an outline of a person. An indicator 604 is displayed on the two-dimensional representation 602 at the determined location of the probe at the time the ultrasound images 606 being displayed were acquired. It should be noted that the images 606 may be, for example, 2D, 3D or 4D ultrasound images, which may be simultaneously, concurrently or sequentially displayed. As different images are displayed, the location of the indicator 604 is updated to show the location of the probe relative to the patient corresponding to when the displayed images 606 were acquired. It should be noted that in this embodiment, an orientation indicator 608 is also provided, illustrated as an arrow 610 in a three-dimensional coordinate axis that shows the orientation of the probe in three-dimensions. It should be noted that in some embodiments, the representation of the patient and probe are both displayed in three-dimensions, such that the probe location and orientation with respect to the patient is ascertainable.

It also should be noted that the indicator 604 may be any shape or size and in some embodiments has a general shape of a probe. Also, the indicator 604 may be sized to indicate the location of the probe within a predetermined zone or region, such as within an area inside a displayed circle to account for some variances in location calculations.

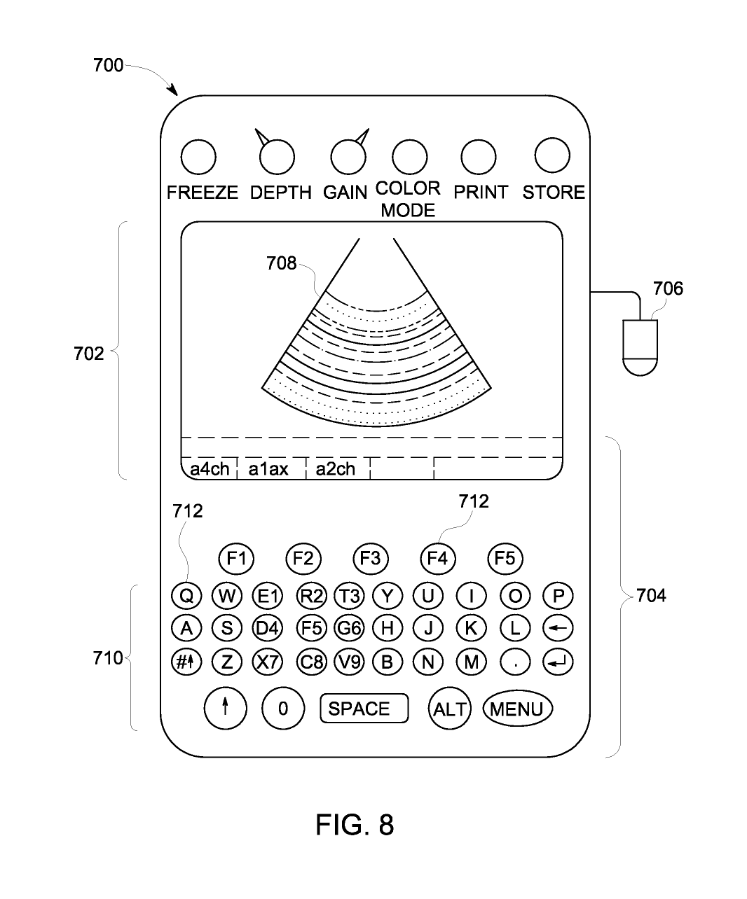

The various embodiments may be implemented in connection with different imaging systems, such as different ultrasound imaging systems. For example, FIG. 8 illustrates a hand carried or pocket-sized ultrasound imaging system 700 (which may be embodied as part of the image communication system 100 shown in FIG. 1). The ultrasound imaging system 700 may be configured to operate and communicate images and probe location information as described in the method 500 (shown in FIG. 6). The ultrasound imaging system 700 has a display 702 and a user interface 704 formed in a single unit. By way of example, the ultrasound imaging system 700 may be approximately two inches wide, approximately four inches in length, and approximately half an inch in depth. The ultrasound imaging system may weigh approximately three ounces. The ultrasound imaging system 700 generally includes the display 702 and the user interface 704, which may or may not include a keyboard-type interface or touch screen and an input/output (I/O) port for connection to a scanning device, for example, an ultrasound probe 706. The display 702 may be, for example, a 320×320 pixel color LCD display on which a medical image 708 or series of medical images 708 may be displayed. A typewriter-like keyboard 710 of buttons 712 may optionally be included in the user interface 704.

The probe 706 may be coupled to the system 700 with wires, cable, or the like. Alternatively, the probe 706 may be physically or mechanically disconnected from the system 700. The probe 706 may wirelessly transmit acquired ultrasound data to the system 700 directly or through an access point device (not shown), such as an antenna disposed within the system 700.

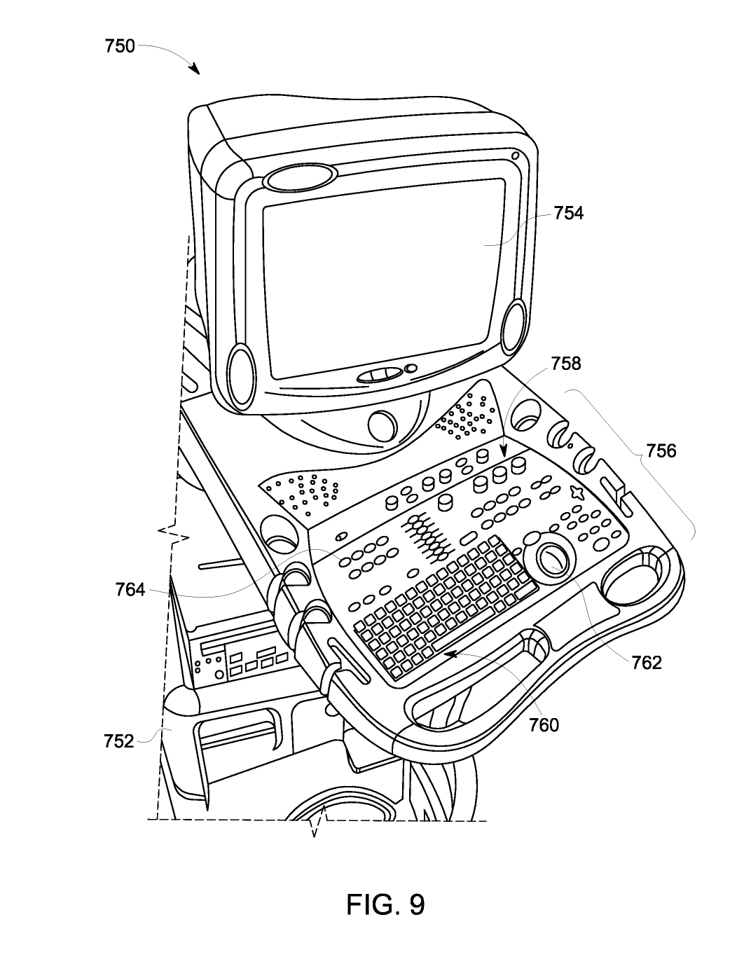

FIG. 9 illustrates an ultrasound imaging system 750 (which may be embodied as part of the image communication system 100) provided on a moveable base 752. The ultrasound imaging system 750 may be configured to operate as described in the method 500 (shown in FIG. 6). A display 754 and a user interface 756 are provided and it should be understood that the display 754 may be separate or separable from the user interface 756. The user interface 756 may optionally be a touchscreen, allowing an operator to select options by touching displayed graphics, icons, and the like.

The user interface 756 also includes control buttons 758 that may be used to control the system 750 as desired or needed, and/or as typically provided. The user interface 756 provides multiple interface options that the user may physically manipulate to interact with ultrasound data and other data that may be displayed, as well as to input information and set and change scanning parameters and viewing angles, etc. For example, a keyboard 760, trackball 762, and/or other controls 764 may be provided. One or more probes (such as the probe 130 shown in FIG. 1) may be communicatively coupled with the system 750 to transmit acquired ultrasound data to the system 750.

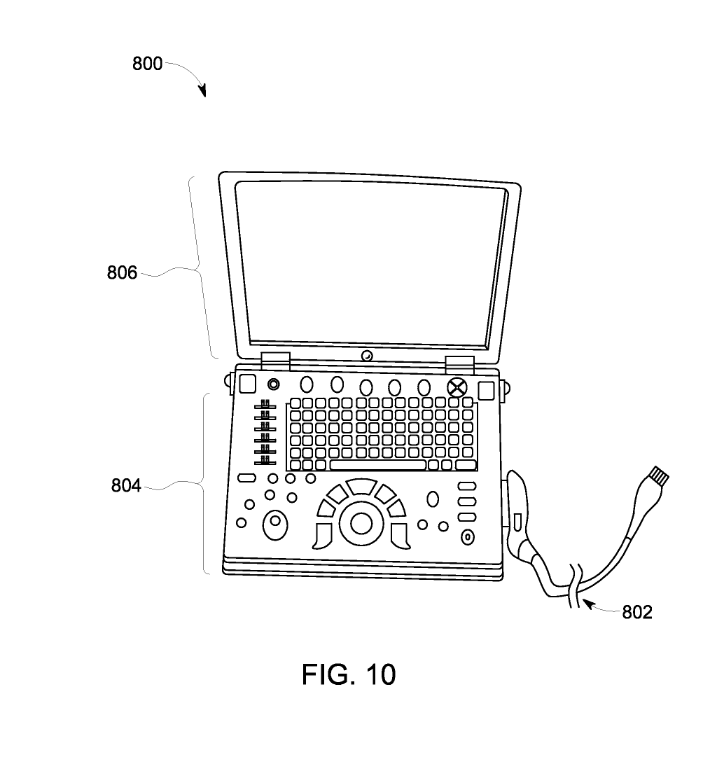

FIG. 10 illustrates a 3D-capable miniaturized ultrasound system 800 (which may be embodied as part of the image communication system 100). The ultrasound imaging system 800 may be configured to operate as described in the method 500 shown in FIG. 6). The ultrasound imaging system 800 has a probe 802 that may be configured to acquire 3D ultrasonic data or multi-plane ultrasonic data. A user interface 804 including an integrated display 806 is provided to receive commands from an operator. As used herein, “miniaturized” means that the ultrasound system 800 is a handheld or hand-carried device or is configured to be carried in a person's hand, pocket, briefcase-sized case, or backpack. For example, the ultrasound system 800 may be a hand-carried device having a size of a typical laptop computer. The ultrasound system 400 is easily portable by the operator, such as in locations remote from a hospital or major health care facility. The integrated display 806 (e.g., an internal display) is configured to display, for example, one or more medical images.

Thus, one or more embodiments may provide transmission of image data and probe location information to enable clinically viable examination and diagnosis from different locations.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, flash drive, jump drive, USB drive and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the described subject matter without departing from their scope. While the dimensions and types of materials described herein are intended to define the parameters of the various embodiments, the embodiments are by no means limiting and are exemplary embodiments. Many other embodiments will be apparent to one of ordinary skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable one of ordinary skill in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

1. An ultrasound imaging system, comprising:
an ultrasound device coupled with an ultrasound probe and configured to acquire ultrasound images of a subject;
at least one of a plurality of digital cameras or a plurality of digital scanners configured to acquire scene information including images of the ultrasound probe with the subject during an image scan; and
a processor having an ultrasound registration unit (URU), the URU configured to identify and reference a probe location of the ultrasound probe to a surface of the object from the scene information and correlate the probe location to one or more of the acquired ultrasound images, the URU further configured to generate a representation of the surface showing the identified and referenced probe location corresponding to the correlated ultrasound images.

2. The ultrasound imaging system of claim 1, wherein the URU is further configured to determine a body contour of the object and fit the location of the ultrasound probe to the body contour using the acquired scene information.

3. The ultrasound imaging system of claim 1, wherein the URU is further configured to solve an over-constrained problem to identify and reference the probe location to the surface of the object.

4. The ultrasound imaging system of claim 1, comprising only a plurality of digital cameras.

5. The ultrasound imaging system of claim 1, comprising only a plurality of digital scanners.

6. The ultrasound imaging system of claim 1, comprising a plurality of digital cameras and a plurality of digital scanners.

7. The ultrasound imaging system of claim 1, further comprising a plurality of retro-reflective patches coupled to the object and the plurality of digital scanners configured to generate a light pattern to illuminate the retro-reflective patches.

8. The ultrasound imaging system of claim 1, further comprising a display remote from the ultrasound device and having a user interface showing the representation of the object with an indicator of the probe location and an orientation of the ultrasound probe corresponding to one or more images being displayed.

9. The ultrasound system of claim 1, wherein the URU is further configured to use outputs from the plurality of digital cameras or the plurality of digital scanners to generate a best fit for the representation of the surface according to a specified norm function.

10. The ultrasound system of claim 1, further comprising a location sensor coupled with the ultrasound probe.

11. A non-transitory computer readable storage medium for identifying an ultrasound probe location corresponding to acquired ultrasound images using a processor, the non-transitory computer readable storage medium including instructions to command the processor to:
obtain ultrasound image data for a subject acquired by the ultrasound probe;
obtain scene information acquired by at least one of a plurality of digital cameras or a plurality of digital scanners, the scene information including images of the ultrasound probe with the subject during an image scan;
identify and reference a probe location of the ultrasound probe to a surface of the object from the scene information and correlate the probe location to one or more of the acquired ultrasound images; and
generate a representation of the surface showing the identified and referenced probe location corresponding to the correlated ultrasound images.

12. The non-transitory computer readable storage medium of claim 11, wherein the instructions command the processor to determine a body contour of the object and fit the location of the ultrasound probe to the body contour using the acquired scene information.

13. The non-transitory computer readable storage medium of claim 11, wherein the instructions command the processor to solve an over-constrained problem to identify and reference the probe location to the surface of the object.

14. The non-transitory computer readable storage medium of claim 11, wherein the instructions command the processor to obtain location information for a plurality of retro-reflective patches coupled to the object acquired by the plurality of digital scanners.

15. The non-transitory computer readable storage medium of claim 11, wherein the instructions command the processor to display remote from the ultrasound device a representation of the object with an indicator of the probe location and an orientation of the ultrasound probe corresponding to one or more images being displayed.

16. The non-transitory computer readable storage medium of claim 11, wherein the instructions command the processor to use outputs from the plurality of digital cameras or the plurality of digital scanners to generate a best fit according to a specified norm function.

17. A method for communicating probe location information synchronized with ultrasound image data, the method comprising:
obtaining ultrasound image data for a subject acquired by an ultrasound probe;
obtaining scene information acquired by at least one of a plurality of digital cameras or a plurality of digital scanners, the scene information including images of the ultrasound probe with the subject during an image scan;
identifying and referencing a probe location of the ultrasound probe to a surface of the object from the scene information and synchronizing in time the probe location to one or more of the acquired ultrasound images;
generating a representation of the surface showing the identified and referenced probe location corresponding to the synchronized ultrasound images; and
communicating the representation with the synchronized ultrasound images to a location remote from an ultrasound system controlling the ultrasound probe.

18. The method of claim 17, further comprising displaying the representation and synchronized ultrasound images at the remote location with an indicator of the probe location and an orientation of the ultrasound probe corresponding to one or more images being displayed and receiving at the ultrasound system feedback from a user at the remote location.

19. The method of claim 17, further comprising determining a body contour of the object and fitting the location of the ultrasound probe to the body contour using the acquired scene information.

20. The method of claim 17, further comprising using outputs from the plurality of digital cameras or the plurality of digital scanners to generate a best fit according to a specified norm function.
